SEQUENCE LISTING (14 sequences)

SEQ ID NO: 1 - DNA, 1866 bp, Rep nucleotide sequence (adeno-associated virus 2)
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 2 - DNA, 2559 bp, Cap nucleotide sequence (adeno-associated virus 2)
cagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 3 - protein, 735 aa, Cap amino acid sequence (adeno-associated virus 2) [sequence omitted]

SEQ ID NO: 4 - DNA, 16 bp, Artificial Sequence
  Rep binding site (RBS)
gcccgagtgagcacgc

SEQ ID NO: 5 - DNA, 350 bp, CARE element (adeno-associated virus 2)
gtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccc

SEQ ID NO: 6 - protein, 137 aa, Artificial Sequence
  L4 22K (Human adenovirus D serotype 9 (HAdV-9))
MPRKKQEPLVEEMEEEWDSQAEEDEWEEETEEEELEEVEEEQATEQPVAAPSAPAAPAVTDTTSAAPAKPPRRWDRVKGDGKHERQGYRSWRAHKAAIIACLQDCGGNIAFARRYLLFHRGVNIPRNVLHYYRHLHS

SEQ ID NO: 7 - DNA, 585 bp, Artificial Sequence
  Ad 5 L4 22K
atggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatag

SEQ ID NO: 8 - protein, 194 aa, Artificial Sequence
  Ad 5 L4 22K
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAGKSKQPPPLAQEQQQRQGYRSWRGHKNAIVACLQDCGGNISFARRFLLYHHGVAFPRNILHYYRHLYSPYCTGGSGSNSSGHTEAKATG

SEQ ID NO: 9 - DNA, 1590 bp, Artificial Sequence
  E2A polypeptide nucleotide sequence (Adenovirus type 5)
atggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 10 - protein, 529 aa, Artificial Sequence
  E2A polypeptide amino acid sequence (Adenovirus type 5)
MASREEEQRETTPERGRGAARRPPTMEDVSSPSPSPPPPRAPPKKRMRRRIESEDEEDSSQDALVPRTPSPRPSTSAADLAIAPKKKKKRPSPKPERPPSPEVIVDSEEEREDVALQMVGFSNPPVLIKHGKGGKRTVRRLNEDDPVARGMRTQEEEEEPSEAESEITVMNPLSVPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 11 - DNA, 19 bp, Artificial Sequence
  TetR binding site
tccctatcagtgatagaga

SEQ ID NO: 12 - DNA, 107 bp, Artificial Sequence
  Modified MLP
cgccctcttcggcatcaaggaaggtgattggtttgtaggtgtaggccacgtgaccgggtgttcctgaaggggggctataaaaggtccctatcagtgatagagactca

SEQ ID NO: 13 - DNA, 107 bp, Artificial Sequence
  Modified MLP
cgccctcttcggcatcaaggaaggtgattggtttgtaggtgtaggccacgtgactccctatcagtgatagagaactataaaaggtccctatcagtgatagagactca

SEQ ID NO: 14 - DNA, 107 bp, Artificial Sequence
  Nucleotide sequence of the wild-type Ad5 MLP
cgccctcttcggcatcaaggaaggtgattggtttgtaggtgtaggccacgtgaccgggtgttcctgaaggggggctataaaagggggtgggggcgcgttcgtcctca

(19) United States
(12) Patent Application Publication
(10) Pub. No.: US 2023/0076955 A1
(43) Pub. Date: Mar. 9, 2023

(54) DNA Amplification Method

(71) Applicants: Oxford Genetics Limited, Oxford Oxfordshire (GB); Oxford University Innovation Limited, Oxford (GB)

(72) Inventors: Ryan Cawood, Oxford Oxfordshire (GB); Weiheng Su, Oxford Oxfordshire (GB)

(21) Appl. No.: 17/796,334

(22) PCT Filed: Feb. 3, 2021
(86) PCT No.: PCT/GB2021/050237

§ 371 Date: Jul. 29, 2022

(30) Foreign Application Priority Data
Feb. 4, 2020 (GB) 2001484.1

(51) Int. Cl. C12Q 1/6844; C12N 15/86; C12N 7/00

(52) U.S. Cl. CPC C12Q 1/6844; C12N 15/86; C12N 7/00; C12N 2710/10041

(57) ABSTRACT

The present invention relates to a method of amplifying a DNA molecule which is operably-linked to a CARE element in a host cell. The method comprises the step of culturing a host cell which comprises a CARE element operably-linked to the DNA molecule, a nucleotide sequence encoding a L4 22K polypeptide or a variant thereof, a nucleic acid molecule comprising a nucleotide sequence encoding an AAV Rep polypeptide or a variant thereof, and optionally one or more further nucleic acid molecules. The invention also relates to nucleic acid molecules encoding a L4 22K polypeptide or a variant thereof, operably-linked to a heterologous promoter; nucleic acid molecules encoding a CARE element operably-linked to viral genes; processes for producing adenoviral vectors and host cells; and processes for producing viral particles, more preferably AAV particles, in host cells.

### CROSS-REFERENCE

This application is a 371 U.S. national phase of PCT/GB2021/050237, filed Feb. 3, 2021, which claims priority from GB application no. 2001484.1, filed Feb. 4, 2020, both which are incorporated by reference in its entirety.

### STATEMENT REGARDING SEQUENCE LISTING

A computer readable form of the Sequence Listing is filed with this application by electronic submission and is incorporated into this application by reference in its entirety. The sequence listing submitted herewith is contained in the file created Jul. 28, 2022, entitled “22-1109-WO-US_Sequence_Listing_ST25.txt” and 24.6 kilobytes in size.

### FIELD OF THE INVENTION

The present invention relates to a method of amplifying a DNA molecule which is operably-linked to a CARE element in a host cell. The method comprises the step of culturing a host cell which comprises a CARE element operably-linked to the DNA molecule, a nucleotide sequence encoding a L4 22K polypeptide or a variant thereof, a nucleic acid molecule comprising a nucleotide sequence encoding an AAV Rep polypeptide or a variant thereof, and optionally one or more further nucleic acid molecules.

The invention also relates to nucleic acid molecules encoding a L4 22K polypeptide or a variant thereof, operably-linked to a heterologous promoter; nucleic acid molecules encoding a CARE element operably-linked to viral genes; processes for producing adenoviral vectors and host cells; and processes for producing viral particles, more preferably AAV particles, in host cells.

### BACKGROUND OF THE INVENTION

Adeno-associated viruses (AAVs) are single-stranded DNA viruses that belong to the Parvoviridae family. This virus is capable of infecting a broad range of host cells, including both dividing and non-dividing cells. In addition, it is a non-pathogenic virus that generates only a limited immune response in most patients.

Over the last few years, vectors derived from AAVs have emerged as an extremely useful and promising mode of gene delivery. This is owing to the following properties of these vectors:
AAVs are small, non-enveloped viruses and they have only two native genes (rep and cap). Thus, they can be easily manipulated to develop vectors for different gene therapies. This is achieved by the removal of the rep and cap genes in the AAV genome and replacing these sequences with exogenous sequences that may provide therapeutic benefit to a patient.
AAV particles are not easily degraded by shear forces, enzymes or solvents. This facilitates easy purification and final formulation of these viral vectors.
AAVs are non-pathogenic and have a low immunogenicity. The use of these vectors further reduces the risk of adverse inflammatory reactions. Unlike other viral vectors, such as lentivirus, herpes virus and adenovirus, AAVs are harmless and are not thought to be responsible for causing any human disease.
Genetic sequences up to approximately 4500 bp can be delivered into a patient using AAV vectors.
Whilst wild-type AAV vectors have been shown to sometimes insert genetic material into human chromosome 19, this property is generally eliminated from most AAV gene therapy vectors by removing rep and cap genes from the viral genome. In such cases, the virus remains in an episomal form within the host cells. These episomes remain intact in non-dividing cells, while in dividing cells they are lost during cell division.

The native AAV genome comprises two genes each encoding multiple open reading frames (ORFs): the rep gene encodes non-structural proteins that are required for the AAV life-cycle and site-specific integration of the viral genome; and the cap gene encodes the structural capsid proteins.

In addition, these two genes are flanked by inverted terminal repeat (ITR) sequences consisting of 145 bases that have the ability to form hairpin structures. These hairpin sequences are required for the primase-independent synthesis of a second DNA strand and the integration of the viral DNA into the host cell genome.

In order to eliminate any integrative capacity of the virus, recombinant AAV vectors remove rep and cap from the DNA of the viral genome. To produce such vectors, the desired transgene(s), together with a promoter(s) to drive transcription of the transgene(s), is inserted between the inverted terminal repeats (ITRs); and the rep and cap genes are provided in trans. Helper genes such as adenovirus E4, E2a and VA genes, are also provided. rep, cap and helper genes may be provided on additional plasmids that are transfected into cells.

Traditionally, the production of AAV vectors has been achieved through a number of different routes. Initially, AAV was generated using wild-type (WT) Adenovirus serotype 5 whilst transfecting cells with plasmids encoding the rep and cap genes and the AAV genome. This allowed the WT adenovirus to provide a number of factors in trans that facilitated virus replication. However, there are a number of limitations to this approach: for example, each batch of AAV must be separated from the Ad5 particles after manufacture to provide a pure product and ensuring that all Ad5 has been removed is challenging. Moreover, the fact that during production the cell is devoting huge resource to the production of Adenoviral particles rather than AAV is also undesirable.

In other systems, stable packing cells lines expressing the rep and cap genes have been used. In such systems, the rep and cap genes are integrated into the cell genomes, hence obviated the need to plasmid-based rep and cap genes. However, these genes are usually only integrated at low frequency (e.g. 1-2 copies per cell) due to their inherent toxicity. These systems require the infection with adenoviral vectors.

More recently, the adenovirus-based systems have been replaced with plasmids encoding the sections of the Adenovirus genome required for AAV production. Whilst this has solved some of the concerns over Adenovirus particles being present in the final virus preparation, a number of issues remain. These include the requirement to pre-manufacture sufficient plasmid for transfection into the production cell line and the inherently inefficient process of transfection itself. The yields from these systems are also lower than those using Ad5-based approaches.

It has previously been reported (Tessier, J., et al. J. Virol. 2001; 375-383; Chadeuf, G., et al. J. Gene Med. 2000; 2:260-268) that the transfection of Hela cell lines into which the rep and cap genes had been chromosomally integrated with AAV vectors and adenoviral infection lead to a 100-fold amplification of the integrated rep-cap sequence. These amplified sequences were present in an extrachromosomal form. An adenovirus DNA-binding protein (DBP) was said to be essential for this amplification.

This phenomenon was further described in US2004/0014031 in which a cis-acting replication element (CARE) was identified. This CARE element was said to be located in a 171 nucleotide region corresponding to nucleotides 190-361 of the AAV-2 genome (Example 12); this encompasses the AAV p5 promoter. This CARE element was said to be bound by a “CARE-dependent replication inducer” (“CARE-DRI”). Such inducers were said to include (whole) adenovirus and herpesvirus. More specifically, the DNA-binding protein (DBP) encoded by the adenovirus Eta gene was identified as a specific inducer of the CARE-dependent amplification of rep and cap genes (US2004/0014031, Example 4).

Additionally, it was reported in US2004/0014031 that the CARE element was capable of inducing the amplification of an adjacent heterologous gene (Example 11), i.e. the CARE element was capable of acting as an origin of replication.

### SUMMARY OF THE INVENTION

It has now been found that the description of the “CARE-dependent replication inducer (CARE-DRI)” in US2004/0014031 as being necessary and sufficient for CARE-dependent replication is incorrect. In US2004/0014031, the inducer is described as the adenoviral DNA binding protein (DBP), a gene product of the E2a expression cassette. It has now been found that, whilst the DBP might be involved in CARE-dependent replication, it is not sufficient to mediate the effect. A product of one of the adenoviral Late genes, i.e. L4 22K, is fundamentally required. This 22K protein was previously known only as being involved in virus encapsidation.

The identification of this specific inducer and its precise mechanism of action facilitates the production of novel methods of amplifying genes to which the CARE element has been juxtaposed, i.e. by the supply of L4 22K polypeptide.

It is one object of the invention, therefore, to provide a method of amplifying a gene of interest to which a CARE element has been juxtaposed.

In one of the Applicant's earlier patent publications (WO2019/020992, the contents of which are specifically incorporated herein in their entirety), the Applicant disclosed that transcription of the Late adenoviral genes could be regulated (e.g. inhibited) by the insertion of a repressor element into the Major Late Promoter. By “switching off” expression of the adenoviral Late genes, the cell's protein-manufacturing capabilities could be diverted toward the production of a desired recombinant protein or AAV particles.

The ability to “switch off” the production of adenoviral Late (i.e. structural) proteins means that no or essentially no adenoviral particles are produced during this process. Consequently, economic savings could be made due to a reduction in the need to remove adenoviral particles from the purified products.

In particular, that invention also had the potential of providing a simple, cost-effective, way to manufacture AAV particles where the Rep and Cap proteins of AAV were integrated and encoded within the genome of a cell to provide the high expression levels which are required to make the AAV particles by maintaining the replication of the Adenoviral genome, but also preventing the production Adenovirus particles in the final AAV preparation.

The Applicants subsequently found, however, that inhibiting the Late adenoviral genes by repressing the Major Late Promoter in the manner described in WO2019/020992 had the undesirable effect of inhibiting DNA amplification of the rep and cap genes from the host cell via inhibition of the CARE-dependent replication mechanism. This was result was entirely unexpected because there are no reports that adenoviral Late gene products are involved in CARE-dependent replication.

The identification of the L4 22K polypeptide as the CARE element induction factor thus enables the use of an AAV production system which utilises the invention described in WO2019/020992, wherein the L4 22K polypeptide is supplied in cis or in trans.

It is thus a further object of the invention to provide a method of AAV production wherein high expression levels of Rep and Cap polypeptides are obtained to make AAV particles, whilst also inhibiting or preventing the production of adenovirus particles.

### BRIEF DESCRIPTION OF THE DRAWINGS

FIG. 1: Adenovirus L4-22K expression is required for production of AAV2 vectors from HelaRC32 cell lines.

FIG. 2: Super infection with TERA-E1 is unable to induce AAV2 production from stable packaging cell line HelaRC32.

FIG. 3: Transcription of L4-22K from adenovirus is required for DNA amplification of stably integrated AAV Rep and Cap genes.

FIG. 4: siRNA knockdown of adenovirus late transcript L4, encoding adenovirus 22K, inhibit AAV2 replication in stable packaging cell line HelaRC32

FIG. 5 Adenovirus late protein L4-22K induce CARE-dependent amplification of AAV Cap genes from HelaRC32 cells.

FIG. 6: Adenovirus late protein L4-22K induces CARE-dependent amplification of AAV Cap genes from HelaRC32 cells in the absence of L4-100K.

### DETAILED DESCRIPTION OF THE INVENTION

In one embodiment, the invention provides a method of amplifying a DNA molecule in a host cell, wherein the DNA molecule is operably-linked to a CARE element, the method comprising the step of culturing a host cell which comprises:
(a) a first nucleic acid molecule comprising the DNA molecule operably-linked to a CARE element;
(b) a second nucleic acid molecule comprising a heterologous promoter operably-associated with a nucleotide sequence encoding a L4 22K polypeptide or a variant thereof;
(c) a third nucleic acid molecule comprising a nucleotide sequence encoding an AAV Rep polypeptide or a variant thereof;
and optionally additionally
(d) one or more further nucleic acid molecules comprising one or more promoters operably-associated with one or more adenovirus Early gene products,
   under conditions such that the second and third, and optionally additionally one or more of the further nucleic acid molecules, are expressed, thus promoting the amplification of the DNA molecule.

Preferably, the one or more adenovirus Early gene products are selected from E2A, VA RNA and E4 gene products.

The first, second, third and (when present) further nucleic acid molecules are preferably present in the host cell:
(i) in an adenoviral vector;
(ii) stably integrated into the host cell genome; or
(iii) in an episomal vector or plasmid.

In another embodiment, the invention provides a process for producing virus particles, the process comprising the steps:
(a) introducing, into a host cell, an adenoviral vector comprising:
   (i) a second nucleic acid molecule of the invention comprising a heterologous promoter operably-associated with a nucleotide sequence which encodes the L4 22K polypeptide or a variant thereof;
   (ii) a Transfer Plasmid comprising 5′- and 3′-viral ITRs flanking a transgene;
   (iii) sufficient helper genes for packaging a viral Transfer Plasmid,
the host cell comprising:
a CARE element, operably-linked to
(i) an AAV cap gene; and
(ii) a nucleic acid molecule comprising a nucleotide sequence encoding a viral Rep polypeptide, preferably wherein the nucleotide sequence is not operably-associated with a functional promoter,
   (b) culturing the host cell under conditions such that virus particles are assembled within the host cell; and
   (c) harvesting packaged virus particles from the cell or the culture medium.

Preferably, the host cell is a viral packaging cell. Preferably, the virus is an AAV.

In another embodiment, the invention provides a process for producing virus particles, the process comprising the steps:
(a) introducing, into a host cell, an adenoviral vector comprising:
   (i) a second nucleic acid molecule of the invention comprising a heterologous promoter operably-associated with a nucleotide sequence which encodes the L4 22K polypeptide or a variant thereof;
   (ii) a nucleic acid molecule comprising a nucleotide sequence encoding an viral Rep polypeptide, preferably wherein the nucleotide sequence is not operably-associated with a functional promoter,
   (iii) sufficient helper genes for packaging a viral Transfer Plasmid,
the host cell comprising:
   (i) a CARE element, operably-linked to an AAV cap gene; and
   (ii) a Transfer Plasmid comprising 5′- and 3′-viral ITRs flanking a transgene, wherein the Transfer Plasmid may or may not be operably-linked to the CARE element,
stably integrated into the host cell genome;
   (b) culturing the host cell under conditions such that virus particles are assembled within the host cell; and
   (c) harvesting packaged virus particles from the host cell or from the culture medium.

In some preferred embodiments, the AAV cap gene is integrated into the host cell genome under the control of a promoter that is activated by a polypeptide that is encoded within the adenoviral vector.

The DNA molecule which is operably-linked to the CARE element may, in general, be any DNA molecule which is desired to be amplified.

CARE amplification may be bi-directional. The DNA molecule may therefore be located 5′ or 3′ to the CARE element. In some embodiments, the length of the nucleotide sequence from the 3′-end of the CARE element to the 3′-end of the DNA molecule is 1-5 Kb, 5-10 Kb, 10-15 Kb, 15-50 Kb or 50-100 Kb. In other embodiments, the length of the nucleotide sequence from the 5′-end of the CARE element to the 5′-end of the DNA molecule is 1-5 Kb, 5-10 Kb, 10-15 Kb, 15-50 Kb or 50-100 Kb.

The DNA molecule may be a coding or non-coding sequence. It may be genomic DNA or cDNA. Preferably, the DNA sequence encodes a polypeptide or a fragment thereof. Preferably, the DNA molecule is operably-associated with one or more transcriptional and/or translational control elements (e.g. an enhancer, promoter, terminator sequence, etc.).

In some embodiments, the DNA molecule codes for a therapeutic polypeptide or a fragment thereof. Examples of preferred therapeutic polypeptides include antibodies, CAR-T molecules, scFV, BiTEs, DARPins and T-cell receptors. In some embodiments, the therapeutic polypeptide is a G-protein coupled receptor (GPCR), e.g. DRD1. In some embodiments, the therapeutic polypeptide is a functioning copy of a gene involved in human vision or retinal function, e.g. RPE65 or REP. In some embodiments, the therapeutic polypeptide is a functioning copy of a gene involved in human blood production or is a blood component, e.g. Factor IX, or those involved in beta and alpha thalassemia or sickle cell anemia. In some embodiments, the therapeutic polypeptide is a functioning copy of a gene involved in immune function such as that in severe combined immune-deficiency (SCID) or Adenosine deaminase deficiency (ADA-SCID).

In some embodiments, the therapeutic polypeptide is a protein which increases/decreases proliferation of cells, e.g. a growth factor receptor. In some embodiments, the therapeutic polypeptide is an ion channel polypeptide. In some preferred embodiments, the therapeutic polypeptide is an immune checkpoint molecule. Preferably, the immune checkpoint molecule is PD1, PDL1, CTLA4, Lag1 or GITR.

In some preferred embodiments, the DNA molecule encodes a CRISPR enzyme (e.g. Cas9, dCas9, Cpf1 or a variant or derivative thereof) or a CRISPR sgRNA.

In some embodiments, the DNA molecule comprises a gene from a virus which is known to infect a mammal. Genes encoded within the DNA molecule may encode polypeptides that are able to self-assemble into viral like particles that may or may not be used as a vaccine. In one preferred embodiment, the DNA molecule encodes a norovirus capsid protein.

In other embodiments, the DNA molecule may encode one or more polypeptides known to induce an immune response in humans as a vaccine that can self-assemble into multimeric complexes. A preferred embodiment would be to encode the five genes required for the cytomegalovirus (CMV) pentameric complex; these include CMV gH/gL/UL128/UL130/UL131.

In other embodiments the genes may encode proteins known to induce an immune response in humans as a vaccine that do not self-assemble into viral like particles. A preferred embodiment would be to encode the Ebola F protein, Influenza F and H proteins or the Coronavirus S, E or M proteins.

In some embodiments, the DNA molecule comprises a gene from a retrovirus, more preferably a lentivirus. Such genes include, but are not limited, to the Gag-Pol gene, the Rev gene, and the Env gene.

In some embodiments, the DNA molecule comprises a gene from a rhabdovirus, more preferably a vesicular stomatitis virus (VSV). Such genes include, but are not limited, to the VSV glycoprotein gene (i.e. the VSV G gene).

In some embodiments, the DNA molecule comprises genes required to make a viral packaging cell line that encodes genes that are required to assemble a gene therapy viral vector or encodes a gene therapy transfer vector.

In some embodiments, the DNA molecule comprises genes required to make a viral producer cell line that encodes all the genes and a transfer vector that are required to produce a gene therapy vector.

In yet other embodiments, the DNA molecule may comprise one or more genes for lentiviral vectors (e.g. Gag-pol, REV, VSV-G, RD114) or one or more genes for adenoviral vectors (e.g. Hexon, Fibre, Penton, pVII, or pVI).

In some embodiments, the DNA molecule comprises a rep gene sequence and/or a cap gene sequence and/or a transfer vector comprising flanking AAV inverted Terminal Repeats (ITRs), or a fragment thereof. Preferably, the rep and cap genes are AAV genes.

In other embodiments, the DNA molecule does not comprise an AAV rep gene sequence or does not comprise an AAV cap gene sequence or does not comprise the sequence of an AAV Inverted Terminal Repeat (ITR), In other embodiments, the DNA molecule does not comprise an AAV sequence. In some embodiments, the CARE element is not linked (contiguously or non-contiguously) to an AAV rep or cap gene.

In some embodiments, the third nucleic acid comprising a nucleotide sequence encoding an AAV Rep polypeptide or a variant thereof is not required.

As used herein, the term “rep gene” refers to a gene that encodes one or more open reading frames (ORFs), wherein each of said ORFs encodes an AAV Rep non-structural protein, or variant or derivative thereof. These AAV Rep non-structural proteins (or variants or derivatives thereof) are involved in AAV genome replication and/or AAV genome packaging.

The wild-type rep gene comprises three promoters: p5, p19 and p40. Two overlapping messenger ribonucleic acids (mRNAs) of different lengths can be produced from p5 and from p19. Each of these mRNAs contains an intron which can be either spliced out or not using a single splice donor site and two different splice acceptor sites. Thus, six different mRNAs can be formed, of which only four are functional. The two mRNAs that fail to remove the intron (one transcribed from p5 and one from p19) read through to a shared poly-adenylation terminator sequence and encode Rep78 and Rep52, respectively. Removal of the intron and use of the 5′-most splice acceptor site does not result in production of any functional Rep protein—it cannot produce the correct Rep68 or Rep40 proteins as the frame of the remainder of the sequence is shifted, and it will also not produce the correct C-terminus of Rep78 or Rep52 because their terminator is spliced out. Conversely, removal of the intron and use of the 3′ splice acceptor will include the correct C-terminus for Rep68 and Rep40, whilst splicing out the terminator of Rep78 and Rep52. Hence the only functional splicing either avoids splicing out the intron altogether (producing Rep78 and Rep52) or uses the 3′ splice acceptor (to produce Rep68 and Rep40). Consequently, four different functional Rep proteins with overlapping sequences can be synthesized from these promoters.

In the wild-type rep gene, the p40 promoter is located at the 3′ end. Transcription of the Cap proteins (VP1, VP2 and VP3) is initiated from this promoter in the wild-type AAV genome.

The four wild-type Rep proteins are Rep78, Rep68, Rep52 and Rep40. Hence the wild-type rep gene is one which encodes the four Rep proteins Rep78, Rep68, Rep52 and Rep40. As used herein, the term “rep gene” includes wild-type rep genes and derivatives thereof; and artificial rep genes which have equivalent functions.

In one embodiment, the rep gene encodes functional Rep78, Rep68, Rep52 and Rep40 polypeptides. In another embodiment, the rep gene encodes functional Rep 78 and Rep 68 polypeptides. In some embodiments, the rep gene p19 promoter is non-functional. In another embodiment, the rep gene encodes non-functional Rep52 and Rep40 polypeptides.

The wild-type AAV (serotype 2) rep gene nucleotide sequence is given in SEQ ID NO: 1.

In one embodiment, the term “rep gene” refers to a nucleotide sequence having at least 70%, 80%, 85%, 90%, 95%, 99% or 100% sequence identity to SEQ ID NO: 1 and which encodes one or more Rep78, Rep68, Rep52 and Rep40 polypeptides.

As used herein, the term “cap gene” refers to a gene that encodes one or more open reading frames (ORFs), wherein each of said ORFs encodes an AAV Cap structural protein, or variant or derivative thereof. These AAV Cap structural proteins (or variants or derivatives thereof) form the AAV capsid.

The three Cap proteins must function to enable the production of an infectious AAV virus particle which is capable of infecting a suitable cell.

The three Cap proteins are VP1, VP2 and VP3, which are generally 87 kDa, 72 kDa and 62 kDa in size, respectively. Hence the cap gene is one which encodes the three Cap proteins VP1, VP2 and VP3.

In the wild-type AAV, these three proteins are translated from the p40 promoter to form a single mRNA. After this mRNA is synthesized, either a long or a short intron can be excised, resulting in the formation of a 2.3 kb or a 2.6 kb mRNA

The AAV capsid is composed of 60 capsid protein subunits (VP1, VP2, and VP3) that are arranged in an icosahedral symmetry in a ratio of 1:1:10, with an estimated size of 3.9 MDa.

As used herein, the term “cap gene” includes wild-type cap genes and derivatives thereof, and artificial cap genes which have equivalent functions. The AAV (serotype 2) cap gene nucleotide sequence and Cap polypeptide sequences are given in SEQ ID NOs: 2 and 3, respectively.

As used herein, the term “cap gene” refers preferably to a nucleotide sequence having the sequence given in SEQ ID NO: 2 or a nucleotide sequence encoding SEQ ID 3: 11; or a nucleotide sequence having at least 70%, 80%, 85% 90%, 95% or 99% sequence identity to SEQ ID NO: 2 or at least 80%, 90%, 95% or 99% nucleotide sequence identity to a nucleotide sequence encoding SEQ ID NO: 3, and which encodes VP1, VP2 and VP3 polypeptides.

The rep and cap genes are preferably viral genes or derived from viral genes. More preferably, they are AAV genes or derived from AAV genes. In some embodiments, the AAV is an Adeno-associated dependoparvovirus A. In other embodiments, the AAV is an Adeno-associated dependoparvovirus B.

11 different AAV serotypes are known. All of the known serotypes can infect cells from multiple diverse tissue types. Tissue specificity is determined by the capsid serotype.

The AAV may be from serotype 1, 2, 3, 4, 5, 6, 7, 8, 9, 10 or 11. Preferably, the AAV is serotype 1, 2, 5, 6, 7, 8 or 9. Most preferably, the AAV serotype is 5 (i.e. AAV5).

The rep and cap genes and each of the protein-encoding ORFs therein) may be from one or more different viruses (e.g. 2, 3 or 4 different viruses). For example, the rep gene may be from AAV2, whilst the cap gene may be from AAV5. It is recognised by those in the art that the rep and cap genes of AAV vary by clade and isolate. The sequences of these genes from all such clades and isolates are encompassed herein, as well as derivatives thereof.

As used herein, the term “CARE” element refers to a Cis-Acting Replication Element. The CARE element is a DNA element which is capable of promoting the replication of an operably-linked DNA molecule. This replication is dependent upon the presence of the adenovirus L4 22K polypeptide or a variant thereof and optionally an E2A polypeptide or variant thereof. Without being bound by theory, it is thought that the L4 22K polypeptide or variant thereof may bind to the CARE element at one or more tttg motifs.

CARE elements comprise a Rep binding site (RBS; gcccgagtgagcacgc SEQ ID NO: 4) and a trs-like element.

The wild-type AAV CARE element comprises the AAV p5 promoter. The TATA box within the CARE element has been shown to be required for CARE amplification. The CARE element is preferably an AAV CARE element.

In the wild-type AAV genome, the CARE element includes the AAV p5 promoter, Rep binding site, the trs element and a 5′ portion of the AAV rep gene. Examples of such CARE elements have previously been described by Tessier, J., et al. J. Virol. 2001; 375-383; Chadeuf, G., et al. J. Gene Med. 2000; 2:260-268; and in US2004/0014031, inter alia. The AAV CARE element is reported to be located between nucleotides 190 to 540 of wild-type AAV2 (Nony, P. et al. J Virol. 2001).

In some preferred embodiment, the CARE element is the 171 nucleotide region corresponding to nucleotides 190-361 of the AAV-2 genome. Preferably, the CARE element has the nucleotide sequence as given in SEQ ID NO: 5 or variant thereof having at least 50%, 60%, 70%, 80%, 90% or 95% sequence identify thereto and which is capable of promoting the amplification of an operably-linked DNA molecule in the presence of a L4 22K polypeptide and optionally an E2A polypeptide.

The binding capacity of a L4 22K polypeptide for a CARE element (or variant thereof) may be assayed by chromatin immunoprecipitation (ChIP) assays. The ability of a L4 22K polypeptide or variant thereof to promote amplification of a DNA molecule which is operably-linked to a CARE element may be assayed by polymerase chain reaction (PCR) or quantitative PCR (as described herein in Examples 3, 5 and 6). In any variant of SEQ ID NO: 5, the sequences of the RBS, TATA box, and trs elements are preferably maintained.

As used herein, the terms “AAV genome”, “AAV Transfer vector” and “Transfer Plasmid” are used interchangeably herein. They all refer to a vector comprising 5′- and 3′-viral (preferably AAV) inverted terminal repeats (ITRs) flanking a transgene.

The CARE element and the DNA molecule are operably-linked. As used herein, the term “operably-linked” (in the context of the CARE element and DNA molecule) means that the CARE element and DNA molecule are linked in a manner such that the CARE element promotes the amplification of the DNA molecule in the presence of a L4 22K polypeptide and optionally additionally an adenoviral E2A polypeptide. This means that the CARE element and the DNA molecule are present in the same DNA molecule, e.g. they are juxtaposed, adjacent or contiguously-linked.

The CARE element may be placed 5′ or 3′ from the DNA molecule to be amplified, preferably 5′. The orientation of the sequence of the CARE element is defined according to its natural (wild-type) environment. The CARE element might be able to function in either the forward or reverse orientation (upstream or downstream relative to the DNA molecule of interest). The distance between the 3′-end of the CARE element and the 5′-end of the DNA molecule is preferably 1 to 1000 nucleotides, more preferably 1-500 nucleotides. In some embodiments, this distance is less than 1000 nucleotides, preferably or less than 50 nucleotides.

The CARE element is contacted within the host cell with a L4 22K polypeptide or a variant thereof, and optionally additionally with an E2A polypeptide or variant thereof.

Adenovirus genes are divided into early (E1-4) and late (L1-5) transcripts, with multiple protein isoforms driven from a range of splicing events.

The early regions are divided into E1, E2, E3 and E4. E1 is essential for transitioning the cell into a phase of the cell cycle that is conducive to virus replication and inhibiting apoptosis and promoting cell division. The E2 region is largely responsible for the replication of the DNA genome. It contains the E2A region which encodes the DNA binding protein (DBP), and the E2B region which primarily encodes the terminal protein, the DNA polymerase (Pol) and the IVa2 protein. E3 contains genes involved in immune regulation of host responses and E4 contains a range of genes involved in regulating cell pathways such as non-homologous end joining (NHEJ) and complexing with E1B-55K to mediate p53 degradation.

The adenovirus late genes are all transcribed from the same promoter, the Major Late Promoter and all share the same 5′ mRNA terminus which contains three exons that collectively form the tri-partite leader sequence. The late genes are expressed by a series of splice events that allow the expression of approximately 13 proteins that either form a part of the virus particle (e.g. Hexon and Fibre) or involved in its assembly (e.g. 100K protein).

The L4 series of transcripts encode the 100K, 33K, 22K, pVII proteins. These proteins are involved in a range of functions. 100K protein is involved in both aiding virus hexon assembly and nuclear import but may also play a role in shifting cell mRNA translation to cap-independent translation. In one embodiment of the invention, the 100K protein may be provided in trans within a cell rather than from within the virus genome. The 22K protein is known to be involved in virus encapsidation. L4 genes are required for successful virus assembly, but not genomic DNA replication.

It has now been found that the L4 22K polypeptide is involved in promoting the amplification of an operably-linked DNA molecule in a CARE-dependent manner.

As used herein, the term “L4 22K polypeptide” refers to the gene product of an adenoviral L4 22K gene, or a variant or derivative thereof. Most preferably, the L4 22K polypeptide is an adenoviral L4 22K polypeptide. The molecular weight of the wild-type adenoviral L4 22K polypeptide is 22 kDa.

Preferably, the adenovirus is a human adenovirus from group A, B, C, D, E, F or G. More preferably, the adenovirus is a human adenovirus from group B or C or D. Even more preferably, the adenovirus is a human adenovirus from groups B or C. Group C is preferred as Ad5 and Ad2 (both group C) are generally used for as helper viruses for AAV manufacture. Ad5 is the most preferred adenovirus.

The human adenovirus D serotype 9 (HAdV-9) L4 22K protein sequence is available from UniProtKB-Q5TJ00. It is given herein in SEQ ID NO: 6. The Ad5 DNA sequence is given herein as SEQ ID NO: 7. The Ad5 amino acid sequence is given in SEQ ID NO: 8.

The nucleotide sequence encoding the L4 22K polypeptide is preferably the nucleotide sequence given in SEQ ID NO: 7 or a nucleotide sequence encoding the polypeptide of SEQ ID NO: 6 or 8; or a variant thereof which has a nucleotide sequence having at least 80%, 85% 90%, 95% or 99% sequence identity to SEQ ID NO: 7 or at least 80%, 90%, 95% or 99% nucleotide sequence identity to a nucleotide sequence encoding the polypeptide of SEQ ID NO: 6 or 8, and which encodes a DNA-binding protein.

Preferably, the L4 22K polypeptide has the amino acid sequence given in SEQ ID NO: 6 or 8, or a variant thereof which has at least 70%, 75%, 80%, 85%, 90%, 95% or 99% amino acid sequence identity with SEQ ID NO: 6 or 8 and which is capable of promoting the amplification of a DNA molecule operably-linked to a CARE element.

In some embodiments, second nucleic acid molecule is provided in the form of a vector or plasmid. The vector or plasmid may be within the host cell (episomally) or introduced into the host cell. In other embodiments, the second nucleic acid molecule is integrated into the host cell genome. In other embodiments, the second nucleic acid molecule is present in a viral vector, e.g. a herpesvirus or lentiviral vector, preferably in an adenoviral vector. The viral vector may be within the host cell or introduced into the host cell.

Most preferably, the second nucleic acid molecule is inserted into an adenoviral vector where the expression of the L4 22K polypeptide or variant thereof is essentially independent of (i.e. not associated with) the adenoviral Major Late Promoter (MLP). The adenoviral vector may be within the host cell or introduced into the host cell.

Preferably, the second nucleic acid molecule of the invention comprising a heterologous promoter operably-associated with a nucleotide sequence encoding a L4 22K polypeptide is located within the adenoviral vector in the E1 or E3 region or an E1/E3-deleted region. It may also be inserted into the L5 region.

The host cell may additionally comprise (d) one or more further nucleic acid molecules comprising one or more promoters operably-associated with one or more adenovirus Early gene products. One or more adenovirus Early gene products may be required in order to enhance the CARE-dependent amplification of the DNA molecule.

In embodiments of the invention which relate to the production of AAVs, one or more adenovirus Early gene products may be required in order to effect the packaging of the AAVs. Preferably, the adenovirus Early gene products are selected from adenoviral E1A, E1B, E2A, VA RNA and E4. These gene products are preferably present within the host cell in an adenoviral vector.

The E2A polypeptide encodes the viral DNA binding protein (DBP). Most preferably, the E2A polypeptide is an adenoviral E2A polypeptide.

Preferably, the adenovirus is a human adenovirus from group A, B, C, D, E, F or G. More preferably, the adenovirus is a human adenovirus from group B or C or D. Even more preferably, the adenovirus is a human adenovirus from groups B or C. Group C is preferred as Ad5 and Ad2 (both group C) are generally used for as helper viruses for AAV manufacture. Ad5 is the most preferred adenovirus.

Preferably, the nucleotide sequence encoding a E2A polypeptide has the sequence given in SEQ ID NO: 9 (Adenovirus type 5). Preferably, the E2A polypeptide has the amino acid sequence given in SEQ ID NO: 10 (Adenovirus type 5).

The nucleic acid molecule encoding a E2A polypeptide is preferably a nucleic acid molecule having the nucleotide sequence given in SEQ ID NO: 9 or a nucleotide sequence encoding SEQ ID NO: 10; or a variant thereof which has a nucleotide sequence having at least 80%, 85% 90%, 95% or 99% sequence identity to SEQ ID NO: 9 or at least 80%, 90%, 95% or 99% nucleotide sequence identity to a nucleotide sequence encoding SEQ ID NO: 10, and which encodes a DNA-binding protein.

Preferably, the E2A polypeptide has the amino acid sequence given in SEQ ID NO: 10 or a variant thereof which has at least 80%, 85%, 90%, 95% or 99% amino acid sequence identity with SEQ ID NO: 10 and which is a DNA-binding protein.

In some embodiments, a nucleic acid molecule encoding a E2A polypeptide is provided in the form of a vector or plasmid. The vector or plasmid may be present within the host cell (episomally) or introduced into the host cell. In other embodiments, a nucleic acid molecule encoding an E2A polypeptide is stably integrated into the host cell genome. In other embodiments, a nucleic acid molecule encoding a E2A polypeptide is present in a viral vector, e.g. a herpesvirus or lentiviral vector, preferably in an adenoviral vector. The viral vector may be present within the host cell or introduced into the host cell.

Preferably, a nucleic acid molecule encoding an E2A polypeptide is provided in an adenoviral vector, more preferably in its native position. Preferably, the nucleic acid molecule encoding an E2A polypeptide is operably-associated with its natural promoter or with a heterologous constitutive promoter.

In some embodiments, the second and further nucleic acid molecules are provided on the same plasmid or vector, or are present in the same viral vector.

In some embodiments, the first nucleic acid molecule and the third nucleic acid molecule are linked such that the nucleotide sequence encoding the AAV Rep polypeptide is operably-linked to the CARE element (and hence the nucleotide sequence encoding the AAV Rep polypeptide is amplified).

The second nucleic acid molecule comprises a heterologous promoter operably-associated with a nucleotide sequence which encodes the L4 22K polypeptide or a variant thereof. As used herein, the term “heterologous promoter” refers to a promoter with which the L4 22K gene is not naturally associated. In wild-type adenoviruses, expression of the L4 22K gene is driven by the Major Late Promoter. The term “heterologous promoter” therefore refers to a promoter which is not an adenoviral Major Late Promoter.

In some embodiments, the heterologous promoter is not an adenoviral promoter, not a herpesvirus promoter or not a viral promoter. In some embodiments, the heterologous promoter is a mammalian promoter. In some embodiments, the heterologous promoter has less than 90%, 80%, 70%, 60% or 50% sequence identify to a wild-type adenoviral Major Later promoter (MLP), preferably that of SEQ ID NO: 14.

The nucleotide sequence of the wild-type Ad5 MLP is given below:

cgccctcttcggcatcaaggaaggtgattggtttgtaggtgtaggccacgtgaccgggtgttcctgaaggggggctataaa agggggtgggggcgcgttcgtcctca (SEQ ID NO: 14)

The TATA box is underlined in the above sequence and the final base (in bold) denotes the position of transcription initiation (i.e. the +1 position).

In some embodiments, the promoter is a constitutive promoter. In other embodiments, the promoter is inducible or repressible. Examples of constitutive promoters include the CMV, SV40, PGK (human or mouse), HSV TK, SFFV, Ubiquitin, Elongation Factor Alpha, CHEF-1, FerH, Grp78, RSV, Adenovirus E1A, CAG or CMV-Beta-Globin promoter, or a promoter derived therefrom. Preferably, the promoter is the cytomegalovirus immediate early (CMV) promoter, or a promoter which is derived therefrom, or a promoter of equal or increased strength compared to the CMV promoter in human cells and human cell lines (e.g. HEK-293 cells).

In some embodiments, the promoter is inducible or repressible by the inclusion of an inducible or repressible regulatory (promoter) element. For example, the promoter may one which is inducible with doxycycline, tetracycline, IPTG or lactose, preferably tetracycline.

In some preferred embodiments, the nucleotide sequence encoding an AAV Rep polypeptide or a rep gene is not operably-associated with a functional promoter. In this way, a low level of expression of Rep polypeptides is obtained, wherein the expression level is sufficiently low such as not to prevent adenoviral growth and not to be sufficiently toxic to cells such as to prevent AAV production.

In the wild-type AAV, expression of the rep gene products are driven by the p5 and p19 promoters. As used herein, the term “the rep gene is not operably-associated with a functional promoter” means that the rep gene does not comprise a functional p5 or a functional p19 promoter, and that the rep gene is not operably-associated with any other functional promoter, such that only baseline or minimal transcription of the rep gene is obtained.

In some preferred embodiments, the AAV cap gene is integrated into the host cell genome under the control of a promoter that is capable of being activated by a polypeptide (an activator) that is encoded within the adenoviral vector.

In yet a further embodiment of the invention, an adenoviral vector of the invention comprises a nucleic acid molecule of the invention which encodes a polypeptide which is capable of transcriptionally-activating a (remote) promoter, for example a promoter which is present in a host cell. Preferably, the promoter in the host cell is one which is operably-associated with (i.e. drives expression of) an AAV cap gene.

In some embodiments, the adenoviral vector encodes a polypeptide which is capable of transcriptionally-activating a promoter which is not present in that adenoviral vector. Examples of such activators include the VP16 transcriptional activator from the herpes simplex virus and the trans-activator domain from the p53 protein. Such activators may be linked to DNA-binding domains such as those that bind to a cumate-binding site or a tetracycline-binding site in the cap gene promoter. This allows transcription of the cap gene only to be induced when the adenoviral vector is present within the host cell, thereby reducing the burden of expressing the AAV cap gene during adenovirus

The host cells may be isolated cells, e.g. they are not situated in a living animal or mammal. Preferably, the host cell is a mammalian cell. Examples of mammalian cells include those from any organ or tissue from humans, mice, rats, hamsters, monkeys, rabbits, donkeys, horses, sheep, cows and apes. Preferably, the cells are human cells. The cells may be primary or immortalised cells.

Preferred cells include HEK-293, HEK 293T, HEK-293E, HEK-293 FT, HEK-293S, HEK-293SG, HEK-293 FTM, HEK-293SGGD, HEK-293A, MDCK, C127, A549, HeLa, CHO, mouse myeloma, PerC6, 911 and Vero cell lines. HEK-293 cells have been modified to contain the E1A and E1B proteins and this obviates the need for these proteins to be supplied on a Helper Plasmid or within an adenoviral vector used in the invention. Similarly, PerC6 and 911 cells contain a similar modification and can also be used. Most preferably, the human cells are HEK293, HEK293T, HEK293A, PerC6 or 911. Other preferred cells include Hela, CHO and VERO cells.

The host cell is cultured (in an appropriate medium) under conditions such that the second, third optionally the further, nucleic acid molecules are expressed. Suitable culture conditions for host cells are well known in the art (e.g. “Molecular Cloning: A Laboratory Manual” (Fourth Edition), Green, M R and Sambrook, J., (updated 2014)). In some embodiments, the host cell will be cultured in a culture medium, preferably a liquid culture medium.

In some embodiments of the invention, the second nucleic acid molecule does not comprise a nucleotide sequence which encodes one or more of the adenoviral L4 33K polypeptide, the adenoviral L4 100K polypeptide or the adenoviral pVIII polypeptide. In some embodiments of the invention, the further nucleic acid molecule does not comprise a nucleotide sequence which encodes the E2B polypeptide. In some embodiments of the invention, the host cell does not comprise an adenovirus or a herpesvirus.

The CARE element is capable of promoting the amplification of the operably-linked DNA molecule. In this regard, the CARE element is acting as an origin of replication. As used herein, the term “amplifying” refers to the production of a plurality of DNA molecules. The plurality of DNA molecules are likely to comprise DNA molecules of different lengths. Each of the DNA molecules in the plurality of DNA molecules will have a nucleotide sequence which comprises all or part of the nucleotide sequence of the CARE element, preferably all of the nucleotide sequence of the CARE element. Each of the DNA molecules in the plurality of DNA molecules will have a nucleotide sequence which comprises all or part of the operably-linked DNA molecule. In some embodiments, the plurality of (amplified) DNA molecules may consist of 50-1000 discrete DNA molecules or more.

The plurality of amplified DNA molecules are double-stranded DNA molecules. The plurality of amplified DNA molecules are linear, extra-chromosomal molecules.

In some embodiments, the method of the invention additionally comprises the step: isolating and/or purifying the amplified DNA molecules and/or the gene products thereof. For example, the amplified DNA products may purified by DNA purification using silica resin in the presence of ethanol. Gene products (e.g. polypeptides) of the amplified DNA products may purified by any method which is suitable for the purification of that particular product, e.g. affinity chromatography.

The DNA molecules, plasmids and vectors of the invention may be made by any suitable technique. Recombinant methods for the production of the nucleic acid molecules and packaging cells of the invention are well known in the art (e.g. “Molecular Cloning: A Laboratory Manual” (Fourth Edition), Green, M R and Sambrook, J., (updated 2014)). The expression of the rep and cap genes, and L4 22K genes, from the DNA molecules of the invention may be assayed in any suitable assay, e.g. by assaying for the number of genome copies per ml by qPCR (as described the Examples herein).

In a further embodiment, the invention provides a method of amplifying a DNA molecule in a host cell, wherein the DNA molecule is operably-linked to a CARE element, the method comprising the step of culturing a host cell which comprises:
(a) a first nucleic acid molecule comprising the DNA molecule operably-linked to a CARE element, wherein the DNA molecule comprises an AAV rep gene and an AAV cap gene;
(b) a second nucleic acid molecule comprising a heterologous promoter operably-associated with a nucleotide sequence encoding a L4 22K polypeptide or a variant thereof;
   and optionally additionally
(c) one or more further nucleic acid molecules comprising one or more promoters operably-associated with one or more adenovirus Early gene products, under conditions such that the second, and optionally additionally one or more of the further nucleic acid molecules, are expressed, thus promoting the amplification of the DNA molecule.

In some embodiments, the second nucleic acid molecule is present in the host cell in an adenoviral vector. In some embodiments, the adenoviral vector additionally comprises an AAV Transfer Plasmid.

In a further embodiment, the invention provides a method of amplifying a DNA molecule in a host cell, wherein the DNA molecule is operably-linked to a CARE element, the method comprising the step of culturing a host cell which comprises:
(a) a first nucleic acid molecule comprising the DNA molecule operably-linked to a CARE element, wherein the DNA molecule comprises a cap gene and optionally additionally an AAV Transfer Plasmid;
(b) a second nucleic acid molecule comprising a heterologous promoter operably-associated with a nucleotide sequence encoding a L4 22K polypeptide or a variant thereof;
(c) a third nucleic acid molecule comprising a nucleotide sequence encoding an AAV Rep polypeptide or a variant thereof;
   and optionally additionally
(d) one or more further nucleic acid molecules comprising one or more promoters operably-associated with one or more adenovirus Early gene products,
   under conditions such that the second and third, and optionally additionally one or more of the further nucleic acid molecules, are expressed, thus promoting the amplification of the DNA molecule.

In some embodiments, the second nucleic acid molecule and/or the third nucleic acid molecule is present in the host cell in an adenoviral vector.

Preferably the rep gene is not operably associated with a functional promoter. Preferably, the rep gene is inserted into the E1 region of an E1/E3-deleted adenoviral vector. Preferably, the rep gene coding sequences are encoded in the same DNA strand as the E2B, E2A and E4 transcription units when positioned in the E1 region.

In a further embodiment, there is provided a process for producing a modified host cell, the process comprising Step (a) and/or Step (b):
(a) introducing a first nucleic acid molecule of the invention into a host cell, wherein the first nucleic acid molecule comprises a DNA molecule which encodes an AAV cap gene operably-linked to a CARE element;
(b) introducing a second nucleic acid molecule of the invention into the host cell;
and optionally:
(c) introducing a third nucleic acid molecule of the invention into the host cell; such that the first, second and (when present) third nucleic acid molecules independently become:
   (i) stably integrated into the genome of the host cell, or
   (ii) present episomally within the host cell.

In some embodiments, the host cell is one which expresses or is capable of expressing the AAV Rep polypeptide and/or Cap polypeptide and/or AAV genome.

For example, the host cell may be one in which one or more DNA molecules comprising nucleotide sequences which encode the AAV Rep polypeptide and/or Cap polypeptide and/or AAV genome are stably integrated. The nucleotide sequences which encode Rep polypeptide and/or Cap polypeptide and/or AAV genome are preferably operably-associated with suitable regulatory elements, e.g. inducible or constitutive promoters.

For example, the host cell may be one which comprises one or more DNA plasmids or vectors comprising nucleotide sequences which encode the AAV Rep polypeptide and/or Cap polypeptide and/or AAV genome. The nucleotide sequences which encode Rep polypeptide and/or Cap polypeptide and/or AAV genome are preferably operably-associated with suitable regulatory elements, e.g. inducible or constitutive promoters. The host cell may be an AAV packaging cell or an AAV producer cell.

In yet a further embodiment, the invention also provides a process for producing a modified adenoviral vector, the process comprising the step of:
(a) introducing a nucleic acid molecule comprising a heterologous promoter operably-associated with a nucleotide sequence encoding a L4 22K polypeptide or a variant thereof into an adenoviral vector;
and optionally
(b) introducing a nucleic acid molecule comprising a nucleotide sequence encoding an AAV Rep polypeptide into an adenoviral vector, wherein, in the adenoviral vector, the nucleic acid molecule encoding the AAV Rep polypeptide is not operably-associated with a functional promoter.

In yet a further embodiment, the invention provides a process for producing viral particles, the process comprising the steps:
(a) introducing a Transfer Plasmid comprising 5′- and 3′-viral ITRs flanking a transgene into a host cell, the host cell comprising:
   (i) a second nucleic acid molecule of the invention comprising a heterologous promoter operably-associated with a nucleotide sequence which encodes the L4 22K polypeptide or a variant thereof;
   (ii) AAV rep and cap genes, the rep and cap genes either being present in an episomal plasmid within the packaging cell or being integrated into the packaging cell genome, the rep and cap genes being operably-linked to a CARE element;
   (iii) sufficient helper genes for packaging the Transfer Plasmid, the helper genes either being present in an episomal Helper Plasmid within the cell, in an adenoviral vector or being integrated into the packaging cell genome;
(b) culturing the host cell under conditions such that viral particles are assembled by the host cell; and
(c) harvesting packaged viral particles from the host cells or from the culture medium.

The culture medium is the medium surrounding the host cells. Preferably, the virus is an AAV. Preferably, the host cell is a viral packaging cell. Preferably, the harvested virus particles are subsequently purified.

The helper genes are preferably selected from one or more of (adenoviral) E1A, E1B, E2A, E4 and VA genes. In some embodiments of the invention, the helper genes additionally include an E2A gene. In other embodiments, the helper genes do not include an E2A gene.

As used herein, the term “introducing” one or more plasmids or vectors into the cell includes transformation, and any form of electroporation, conjugation, infection, transduction or transfection, inter alia. Processes for such introduction are well known in the art (e.g. Proc. Natl. Acad. Sci. USA. 1995 Aug. 1; 92 (16):7297-301).

In some preferred embodiments, the transgene encodes a CRISPR enzyme (e.g. Cas9, Cpf1) or a CRISPR sgRNA. In other embodiments the transgene is a gene involved in haemophilia (e.g. factor VIII or IX).

WO2019/020992 discloses that transcription of the Late adenoviral genes can be regulated (e.g. inhibited) by the insertion of a repressor element into the Major Late Promoter. By “switching off” expression of the adenoviral Late genes, the cell's protein-manufacturing capabilities can be diverted toward the production of a desired recombinant protein or AAV particles. The Applicants subsequently found, however, that inhibiting the Late adenoviral genes by repressing the Major Late Promoter in the manner described in WO2019/020992 had the undesirable effect of inhibiting CARE dependent replication of the rep and cap genes if those genes were integrated into the host cell genome. The identification of the L4 22K polypeptide as the CARE element-inducing polypeptide thus enables the use of an AAV production system which utilises the invention described in WO2019/020992 (the contents of which are specifically incorporated herein in their entirety), wherein the L4 22K polypeptide is supplied in cis or in trans.

In another embodiment, the invention provides a process for producing virus particles, the process comprising the steps:
(a) introducing, into a host cell, an adenoviral vector comprising:
   (i) a second nucleic acid molecule of the invention comprising a heterologous promoter operably-associated with a nucleotide sequence which encodes the L4 22K polypeptide or a variant thereof;
   (ii) a Transfer Plasmid comprising 5′- and 3′-viral ITRs flanking a transgene;
   (iii) sufficient helper genes for packaging a viral Transfer Plasmid, the host cell comprising:
   a CARE element, operably-linked to
   (i) an AAV cap gene; and
   (ii) a nucleic acid molecule comprising a nucleotide sequence encoding a viral Rep polypeptide, preferably wherein the nucleotide sequence is not operably-associated with a functional promoter.
(b) culturing the host cell under conditions such that virus particles are assembled within the host cell; and
(c) harvesting packaged virus particles from the host cells or from the culture medium.

In another embodiment, the invention provides a process for producing virus particles, the process comprising the steps:
(a) introducing, into a host cell, an adenoviral vector comprising:
   (i) a second nucleic acid molecule of the invention comprising a heterologous promoter operably-associated with a nucleotide sequence which encodes the L4 22K polypeptide or a variant thereof;
   (ii) a nucleic acid molecule comprising a nucleotide sequence encoding an viral Rep polypeptide, preferably wherein the nucleotide sequence is not operably-associated with a functional promoter,
   (iii) sufficient helper genes for packaging a viral Transfer Plasmid,
the host cell comprising:
   (i) a CARE element, operably-linked to an AAV cap gene; and
   (ii) a Transfer Plasmid comprising 5′- and 3′-viral ITRs flanking a transgene, wherein the Transfer Plasmid may or may not be operably-linked to the CARE element,
stably integrated into the host cell genome;
   (b) culturing the host cell under conditions such that virus particles are assembled within the host cell; and
   (c) harvesting packaged virus particles from the host cells or from the culture medium.

In some preferred embodiments, the AAV cap gene is integrated into the host cell genome under the control of a promoter that is activated by a polypeptide that is encoded within the adenoviral vector. Preferably, the virus is an AAV. Preferably, the host cell is a viral packaging cell.

Preferably, the adenoviral vector comprises a repressible Major Late Promoter (MLP), more preferably wherein the MLP comprises one or more repressor elements which are capable of regulating or controlling transcription of the adenoviral late genes, and wherein one or more of the repressor elements are inserted downstream of the MLP TATA box.

Preferably, the a CARE element, AAV cap gene; and Transfer plasmid are:
(i) stably integrated into the host cell genome; or
(ii) present in the host cell in an episomal plasmid or vector.

Preferred features of the process for producing viral (preferably AAV) particles include the following:
wherein the one or more repressor elements are inserted between the MLP TATA box and the +1 position of transcription.
wherein the repressor element is one which is capable of being bound by a repressor protein.
wherein a gene encoding a repressor protein which is capable of binding to the repressor element is encoded within the adenoviral genome.
wherein the repressor protein is transcribed under the control of the MLP.
wherein the repressor protein is the tetracycline repressor, the lactose repressor or the ecdysone repressor, preferably the tetracycline repressor (TetR).
wherein the repressor element is a tetracycline repressor binding site comprising or consisting of the sequence set forth in SEQ ID NO: 11.
wherein the nucleotide sequence of the MLP comprises or consists of the sequence set forth in SEQ ID NO: 12 or 13.
wherein the presence of the repressor element does not affect production of the adenoviral E2B protein.
wherein the adenoviral vector encodes the adenovirus L4 100K protein and wherein the L4 100K protein is not under control of the MLP.
wherein a transgene is inserted within one of the adenoviral early regions, preferably within the adenoviral E1 region instead of in a Transfer Plasmid.
wherein the transgene comprises a Tripartite Leader (TPL) in its 5′-UTR.
wherein the transgene encodes a therapeutic polypeptide.
wherein the transgene encodes a virus protein, preferably a protein that is capable of assembly in or outside of a cell to produce a virus-like particle, preferably wherein the transgene encodes Norovirus VP1 or Hepatitis B HBsAG.

In another embodiment, the invention provides a DNA molecule encoding a L4 22 K polypeptide:
(i) wherein the DNA molecule is operably-associated with a promoter which is not an adenoviral Major Late Promoter;
(ii) wherein the DNA molecule is operably-associated with a mammalian promoter;
(iii) wherein DNA molecule does not additionally encode an adenoviral L4 100 K, L4 33K or pVII polypeptide;
(iv) wherein the DNA molecule is operably-associated with a CMV, PGK or SV40 promoter.

In another embodiment, the invention provides a host cell comprising:
(a) a second nucleic acid molecule of the invention comprising a heterologous promoter operably-associated with a nucleotide sequence encoding a L4 22K polypeptide, or a variant thereof,
   wherein the nucleic acid molecule is stably integrated into the host cell's genome or is present in an episomal plasmid or vector.

Preferably,
(i) the heterologous promoter is not an adenoviral Major Late Promoter;
(ii) the promoter is a mammalian promoter;
(iii) the nucleic acid molecule does not additionally encode an adenoviral L4 100K, L4 33K or pVII polypeptide; or
iv) wherein the DNA molecule is operably-associated with a CMV, PGK or SV40 promoter.

Preferably, the host cell is one as defined herein.

In some embodiments, the host cell additionally comprises:
(b) AAV rep and cap genes, the rep and cap genes either being present in an episomal plasmid within the host cell or being stably integrated into the cell genome, the rep and cap genes being operably-linked to a CARE element.

In yet other embodiments, the host cell additionally comprises one or both of:
(c) an AAV Transfer Plasmid comprising a transgene flanked by ITRs; and
(d) an adenoviral Helper Plasmid for AAV production comprising one or more genes selected from E1A, E1B, E2A, E4 and VA RNA.

Such cells are commonly known as packaging cells.

In some embodiments of the invention, the Helper Plasmid additionally comprises an E2A gene. In other embodiments, the Helper Plasmid does not comprise an E2A gene. In the latter case, the omission of the E2A gene reduces considerably the amount of DNA which is needed in the Helper Plasmid.

In another embodiment, the invention provides a host cell comprising:
(a) a first nucleic acid molecule of the invention comprising a DNA molecule operably-linked to a CARE element, wherein the DNA molecule encodes one or more of:
   (i) an AAV Cap polypeptide,
   (ii) an AAV Rep polypeptide and
   (iii) an AAV Transfer vector,
      wherein the first nucleic acid molecule is stably integrated into the host cell's genome or is present in an episomal plasmid.

In yet another embodiment, the invention provides an adenoviral vector comprising a nucleic acid molecule which encodes an adenoviral L4 22K polypeptide or a variant thereof, wherein the L4 22K polypeptide or a variant thereof coding sequence is not operably-associated with the adenoviral MLP.

In some such embodiments, it may be preferable to insert a new L4 22K coding sequence into the adenoviral vector in addition to the native L4 22K coding sequence.

In such embodiments, the adenoviral vector comprises:
(i) a nucleic acid molecule which encodes an adenoviral L4 22K polypeptide or a variant thereof, wherein the L4 22K polypeptide or a variant thereof coding sequence is not operably-associated with the adenoviral MLP; and
(ii) a nucleic acid molecule which encodes an adenoviral L4 22K polypeptide, wherein the L4 22K polypeptide coding sequence is operably-associated with the adenoviral MLP.

Preferably, the adenoviral MLP is a repressible MLP (for example, as defined herein)

Preferably, the adenoviral vector additionally comprises a nucleic acid molecule which encodes an AAV Rep polypeptide, more preferably wherein the nucleic acid molecule is not operably-associated with a functional promoter.

Preferably, the L4 22K polypeptide encoding sequence is inserted into the adenoviral E1 or E3 region.

The invention also provides a kit comprising:
(A) a host cell comprising:
(a) a first nucleic acid molecule of the invention comprising a DNA molecule operably-linked to a CARE element, wherein the DNA molecule encodes one or more of
   (i) an AAV Cap polypeptide, and
   (ii) an AAV Rep polypeptide
      wherein the first nucleic acid molecule is stably integrated into the host cell's genome or is present in an episomal plasmid;
      and
(B) an adenoviral vector comprising:
   (i) a nucleic acid molecule which encodes an adenoviral L4 22K polypeptide or a variant thereof, wherein L4 22K polypeptide or a variant thereof coding sequence is not operably-associated with the adenoviral MLP, and
   (ii) a nucleic acid molecule which encodes an AAV Transfer Vector.

The invention also provides a kit comprising:
(A) a host cell comprising:
(a) a first nucleic acid molecule of the invention comprising a DNA molecule operably-linked to a CARE element, wherein the DNA molecule encodes one or more of
   (i) an AAV Cap polypeptide, and optionally
   (ii) an AAV Transfer Vector,
      wherein the first nucleic acid molecule is stably integrated into the host cell's genome or is present in an episomal plasmid;
      and
(B) an adenoviral vector comprising:
   (i) a nucleic acid molecule which encodes an adenoviral L4 22K polypeptide or a variant thereof, wherein L4 22K polypeptide or a variant thereof coding sequence is not operably-associated with the adenoviral MLP, and
   (ii) a nucleic acid molecule which encodes an AAV Rep polypeptide, preferably wherein the nucleic acid molecule is not operably-associated with a functional promoter.

The kit may also contain materials for purification of AAV particles such as those involved in the density banding and purification of viral particles, e.g. one or more of centrifuge tubes, iodixanol, dialysis buffers and dialysis cassettes.

There are many established algorithms available to align two amino acid or nucleic acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid or nucleic acid sequences for comparison may be conducted, for example, by computer-implemented algorithms (e.g. GAP, BESTFIT, FASTA or TFASTA), or BLAST and BLAST 2.0 algorithms.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. (See Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters.

In other embodiments, a BLAST Global Alignment program may be used (such as that available from the NCBI) using a Needleman-Wunsch alignment of two protein sequences with the gap costs: Existence 11 and Extension 1.

The disclosure of each reference set forth herein is specifically incorporated herein by reference in its entirety.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Adenovirus L4-22K Expression is Required for Production of AAV2 Vectors from HelaRC32 Cell Lines

L4 22K is expressed from the adenovirus major late promoter during late phase of infection and transcriptional repression of the MLP represses AAV2 production from HelaRC32 cells. Control adenovirus Ad5-E1 and TERA-E1 (a recombinant replicating adenovirus wherein its modified major late promoter transcribes the repressor protein TetR, and wherein transcription from the modified major late promoter is repressed by the TetR) were generated by molecular cloning methods and produced from HEK293 cells. HELARC32 cells were seeded in 48-well tissue culture plates at 9e4 cells/well for 24-hours prior to transfection with plasmid pSF-AAV-EGFP and infected, in the presence of doxycycline 0.5 ug/mL or DMSO, with Ad5-E1 or TERA-E1. AAV2 particles were harvested after 96-hours post-production and quantified by QPCR. The results are shown in FIG. 1.

### Example 2: Super Infection with TERA-E1 is Unable to Induce AAV2 Production from Stable Packaging Cell Line HelaRC32

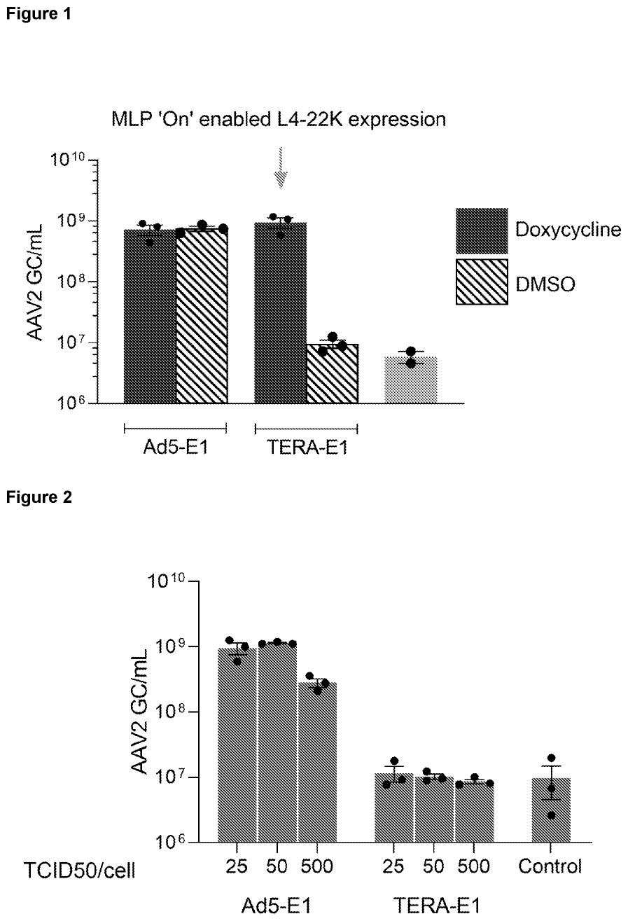

Control adenovirus Ad5-E1 and TERA-E1 (a recombinant replicating adenovirus wherein its modified major late promoter transcribes the repressor protein TetR, and wherein transcription from the modified major late promoter is repressed by the TetR) were generated by molecular cloning methods and produced from HEK293 cells. HeLaRC32 cells were seeded in 48-well tissue culture plates at 9e4 cells/well for 24-hours prior to transfection with plasmid pSF-AAV-EGFP and infected, in the absence of doxycycline 0.5 ug/mL or DMSO, with Ad5-E1 or TERA-E1 at the indicate multiplicity of infection. AAV2 particles were harvested after 96-hours post-production and quantified by QPCR. The results are shown in FIG. 2.

### Example 3: Transcription of L4-22K from Adenovirus is Required for DNA Amplification of Stably Integrated AAV Rep and Cap Genes

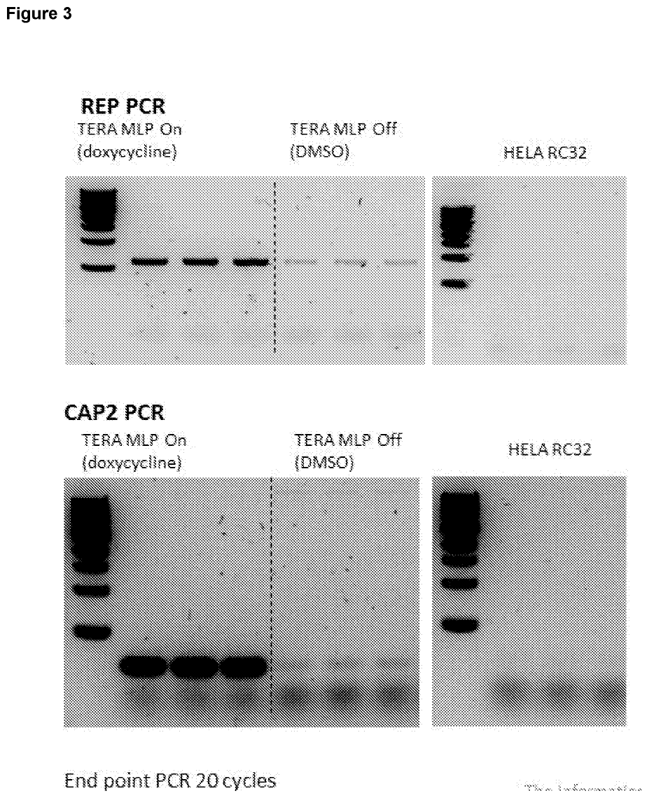

TERA-E1 (a recombinant replicating adenovirus wherein its modified major late promoter transcribes the repressor protein TetR, and wherein transcription from the modified major late promoter is repressed by the TetR) was generated by molecular cloning methods and produced from HEK293 cells. HELARC32 cells were seeded in 48-well tissue culture plates at 9e4 cells/well for 24-hours and infected with TERA-E1 MOl50, in the presence of doxycycline 0.5 ug/mL or DMSO. Total DNA was extracted 96-hours post-infection and AAV Rep and Cap DNA amplified by PCR. AAV Rep and Cap amplicon DNA resolved by agarose gel electrophoreses. The results are shown in FIG. 3.

### Example 4: siRNA Knockdown of Adenovirus Late Transcript L4, Encoding Adenovirus 22K, Inhibit AAV2 Replication in Stable Packaging Cell Line HelaRC32

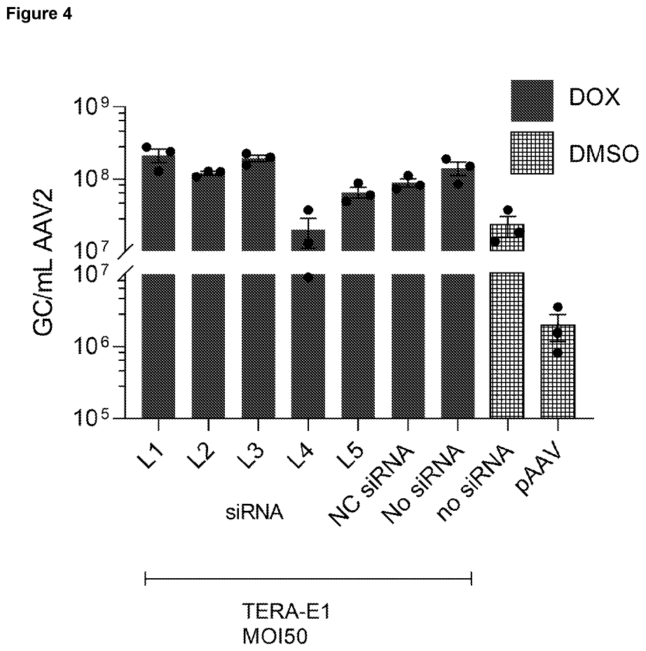

MLP-repressible adenoviruses TERA-E1 (a recombinant replicating adenovirus wherein its modified major late promoter transcribes the repressor protein TetR, and wherein transcription from the modified major late promoter is repressed by the TetR) was generated by standard molecular cloning methods and produced from HEK293 cells. HELARC32 cells were seeded in 48-well tissue culture plates at 1.5e4 cells/well was transfected with siRNA targeting adenovirus primary mRNA transcript L1, L2, L3, L4 or L5 for 24-hours. HelaRC32 cells were transfected with plasmid pSF-AAV-EGFP and infected with TERA-E1 MOl50, in the presence of doxycycline 0.5 ug/mL or DMSO. AAV2 quantified by QPCR 96-hours post infection. The results are shown in FIG. 4.

### Example 5: Adenovirus Late Protein L4-22K Induce CARE-Dependent Amplification of AAV Cap Genes from HelaRC32 Cells

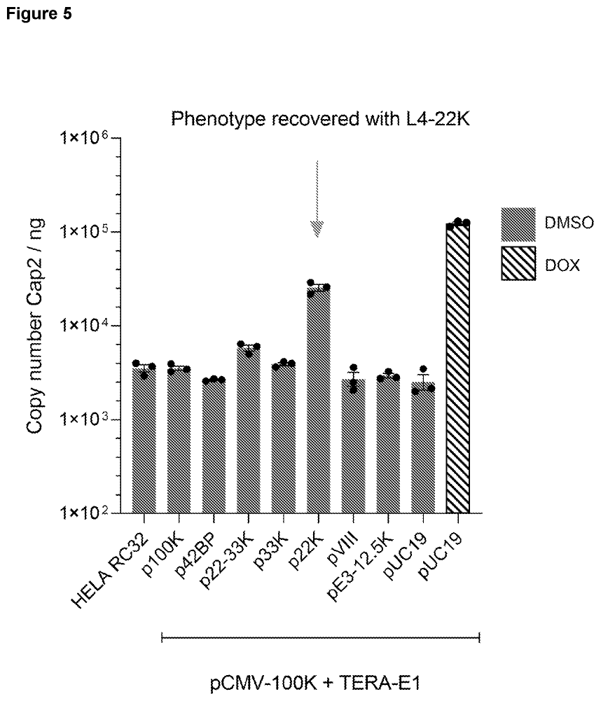

MLP-repressible adenoviruses TERA-E1 (a recombinant replicating adenovirus wherein its modified major late promoter transcribes the repressor protein TetR, and wherein transcription from the modified major late promoter is repressed by the TetR) was generated by molecular cloning methods and produced from HEK293 cells. HeLaRC32 cells were seeded in 48-well tissue culture plates at 9.0e4 cells/well for 24-hours before transfection with plasmids transcribing adenovirus L4 genes under control of the CMV promoter, and infection with TERA-E1 MOl50. Total DNA was extracted 96-hours post-infection and AAV Cap DNA quantified by QPCR. The results are shown in FIG. 5.

### Example 6: Adenovirus Late Protein L4-22K Induces CARE-Dependent Amplification of AAV Cap Genes from HelaRC32 Cells in the Absence of L4-100K

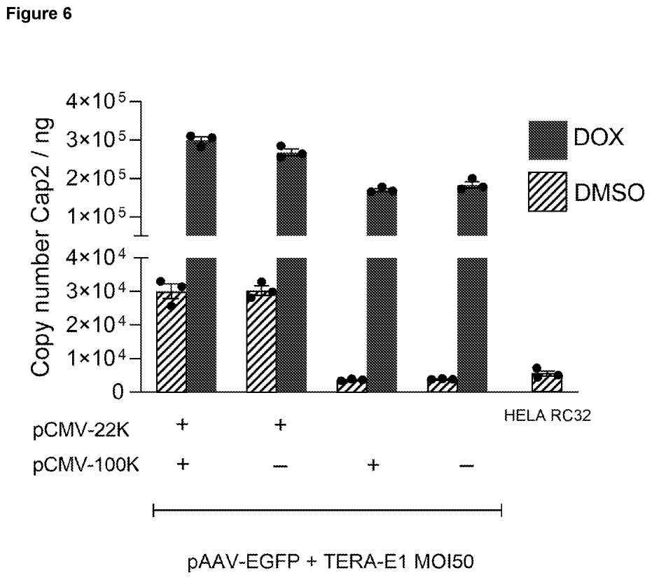

MLP-repressible adenoviruses TERA-E1 (a recombinant replicating adenovirus wherein its modified major late promoter transcribes the repressor protein TetR, and wherein transcription from the modified major late promoter is repressed by the TetR) was generated by molecular cloning methods and produced from HEK293 cells. HeLaRC32 cells were seeded in 48-well tissue culture plates at 9.0e4 cells/well for 24-hours before co-transfection of CMV promoter plasmids transcribing adenovirus L4-22K, with CMV driven L4-100K or stuffer DNA, and infection with TERA-E1 MOl50. Total DNA was extracted 96-hours post-infection and AAV Cap DNA quantified by QPCR. The results are shown in FIG. 6.

### SEQUENCES

Rep nucleotide sequence (AAV serotype 2)
SEQ ID NO: 1
atgccggggttttacgagattgtgattaaggtccccagcgaccttgacgagcatctgcccggcatttctgacagctt
tgtgaactgggtggccgagaaggaatgggagttgccgccagattctgacatggatctgaatctgattgagcaggcac
ccctgaccgtggccgagaagctgcagcgcgactttctgacggaatggcgccgtgtgagtaaggccccggaggccctt
ttctttgtgcaatttgagaagggagagagctacttccacatgcacgtgctcgtggaaaccaccggggtgaaatccat
ggttttgggacgtttcctgagtcagattcgcgaaaaactgattcagagaatttaccgcgggatcgagccgactttgc
caaactggttcgcggtcacaaagaccagaaatggcgccggaggcgggaacaaggtggtggatgagtgctaaatcccc
aattacttgctccccaasacccagcctgagctccagtgggcgtggactaatatggaacagtatttaagcgcctgttt
gaatctcacggagcgtaaacggttggtggcgcagcatctgacgcacgtgtcgcagacgcaggagcagaacaaagaga
atcagaatcccaattctgatgcgccggtgatcagatcaaaaacttcagccaggtacatggagctggtcgggtggcta
gtggacaaggggattacctcggagaagcagtggatccaggaggaccaggcctcatacatctccttcaatgcggcctc
caactcgcggtcccaaatcaaggctgccttggacaatgcgggaaagattatgagcctgactaaaaccgcccccgact
acctggtgggccagcagcccgtggaggacatttccagcaatcggatttataaaattttggaactaaacgggtacgat
ccccaatatgcggcttccgtctttctgggatgggccacgaaaaagttcggcaagaggaacaccatctggctgtttgg
gcctgcaactaccgggaagaccaacatcgcggaggccatagcccacactgtgcccttctacgggtgcgtaaactgga
ccaatgagaactttcccttcaacgactgtgtcgacaagatggtgatctggtgggaggaggggaagatgaccgccaag
gtcgtggagtcggccaaagccattctcggaggaagcaaggtgcgcgtggaccagaaatgcaagtcctcggcccagat
agacccgactcccgtgatcgtcacctccaacaccaacatgtgcgccgtgattgacgggaactcaacgaccttcgaac
accagcagccgttgcaagaccggatgttcaaatttgaactcacccgccgtctggatcatgactttgggaaggtcacc
aagcaggaagtcaaagactttttccggtgggcaaaggatcacgtggttgaggtggagcatgaattctacgtcaaaaa
gggtggagccaagaaaagacccgcccccagtgacgcagatataagtgagcccaaacgggtgcgcgagtcagttgcgc
agccatcgacgtcagacgcggaagcttcgatcaactacgcagacaggtaccsaaacaaatgttctcgtcacgtgggc
atgaatctgatgctgtttccctgcagacaatgcgagagaatgaataagaattcaaatatctgcttcactcacggaca
gaaagactgtttagagtgctttcccgtgtcagaatctcaacccgtttctgtcgtcaaaaaggcgtatcagaaactgt
gctacattcatcatatcatgggaaaggtgccagacgcttgcactgcctgcgatctggtcaatgtggatttggatgac
tgcatctttgaacaaTAG
Cap nucleotide sequence (AAV serotype 2)
SEQ ID NO: 2
Cagttgcgcagccatcgacgtcagacgcggaagcttcgatcaactacgcagacaagtaccaaaacaaatgttctcgt
cacgtgggcatgaatctgatgctgtttccctgcagacaatgcgagagaatgaatcagaattcaaatatctgcttcac
tcacggacagaaagactgtttagagtgctttcccgtgtcagaatctcaacccgtttctgtcgtcaaaaaggcgtatc
agaaactgtgctacattcatcatatcatgggaaacgtgccagacgcttgcactgcctgcgatctggtcaatgtgcat
ttagatgactgcatctttgaacaataaatgatttaaatcaggt
atggctgccgatggttatcttccagattggctcgaggacactctctctgaaggaataagacagtggtggaagctcaa
acctggcccaccaccaccaaagcccgcagagcggcataaggacgacagcaggggtcttgtgcttcctgggtacaagt
acctcgcacccttcaacggactcgacaagggagagccgctcaacgacgcagacgccgcggccctcgagcacgacaaa
gcctacgaccggcagctcgacagcggagacaacccgtacctcaagtacaaccacgccgacgcggagtttcaggagcg
ccttaaagaagatacgtcttttgggggcaacctcggacgagcagtcttccaggcgaaaaagagggttcttgaacctc
tgggcctggttgaggaacctgttaagacggctccgggaaaaaagaggccggtagagcactctcctgtggagccagac
tcctcctcgggaaccggaaaggcgggccagcagcctgcaagaaaaagattgaattttggtcagactggagacgcaga
ctcagtacctgacccccagcctctcggacagccaccagcagccccctctggtctgggaactaatacgatggctacag
gcagtggcgcaccaatggcagacaataacgagggcgccgacggagtgggtaattcctcgggaaattggcattgcgat
tccacatggatgggcgacagagtcatcaccaccagcacccgaacctgggccctgcccacctacaacaaccacctcta
caaacaaatttccagccaatcaggagcctcgaacgacaatcactactttggctacagcaccccttgggggtattttg
acttcaacagattccactgccacttttcaccacgtgactggcaaagactcatcaacaacaactggggattccgaccc
aagagactcaacttcaagctctttaacattcaagtcaaagaggtcacgcagaatgacggtacgacgacgattgccaa
taaccttaccagcacggttcaggtgtttactgactcggagtaccagctcccgtacgtcctcggctcggcgcatcaag
gatgcctcccgccgttcccagcagacgtcttcatggtgccacagtatggatacctcaccctgaacaacgggagtcag
gcagtaggacgctcttcattttactgcctggagtactttccttctcagatgctgcgtaccggaaacaactttacctt
cagctacacttttgaggacgttcctttccacagcagctacgctcacagccagagtctggaccgtctcatgaatcctc
tcatcgaccagtacctgtattacttgagcagaacaaacactccaagtggaaccaccacgcagtcaaggcttcagttt
tctcaggcaggagcgagtgacattagggaccagtctaggaactggcttactggaccatgttaccgccagcagcgagt
atcaaagacatctgcggataacaacaacagtgaatactcgtggactggagctaccaagtaccacctcaatggcagag
actctctggtgaatccgggcccggccatggcaagccacaaggacgatgaagaaaagttttttcctcagagcggggtt
ctcatctttgggaagcaaggctcsgagaaaacaaatgtggacattgaaaaggtcatgattacagacgaagaggaaat
caggacaaccaatcccgtggctacggagcagtatggttctgtatctaccaacctccagagaggcaacagacaagcag
ctaccgcagatgtcaacacacaaggcgttcttccaggcatggtctggcaggacagagatgtgtaccttcaggggccc
atctgggcaaagattccacacacggacggacattttcacccctctcccctcatgggtggattcggacttaaacaccc
tcctccacagattctcatcaagaacaccccggtacctgcgaatccttcgaccaccttcagtgcggcaaagtttgctt
acttcatcacacagtactccacgggacaggtcagcgtggagatcgagtgggagctgcagaaggaaaacagcaaacgc
tggaatcccgaaattcagtacacttccaactacaacaagtctattaatgtagactttactgtggacactaatggcgt
gtattcagagcctcgcccattggcaccagatacctgactcgtaatctgtaA
Cap amino acid sequence (AAV serotype 2)
SEQ ID NO: 3
MAADGYLPDWLEDTLSEGIRQWWKLKPGPPPPKPAERHKDDSRGLVLPGYKYLGPFNGLDKGEPVNEADAAALEHDK
AYDRQLDSGDNPYLKYNHADAEFQERLKEDTSFGGNLGRAVFQAKKRVLEPLGLVEEPVKTAPGKKRPVEHSPVEPD
SSSGTGKAGQQPARKRLNFGQTGDADSVPDPQPLGQPPAAPSGLGTNTMATGSGAFMADNNEGADGVGNSSGNWHCD
STWMGDRVITTSTRTWALPTYNNHLYKQISSQSGASNDNHYFGYSTFWGYFDFNRFECHFSPRDWQRLINNNWGFRP
KRLNFKLFNIQVKEVTQNDGTTTIANNLTSTVQVFTDSEYQLPYVLGSAHQGCLPPFPADVFMVPQYGYLTLNNGSQ
AVGRSSFYCLEYFPSQMLRTGNNFTFSYTFEDVPFHSSYAHSQSLDRLMNFLIDQYLYYLSRTNTPSGTTTQSRLQE
SQAGASDIRDQSENWLPGPCYRQQRVSKTSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNRQAATADVNTQGVLPGMVWQDRDVYLQGP
IWAKIPHTDGHFHPSPLMGGFGLKHPPPQILIKNTPVPANPSTTFSAAKFASFITQYSTGQVSVEIEWELQKENSKR
WNPEIQYTSNYNKSVNVDFTVDTNGVYSEPRFIGTRYLTPNL*
Rep binding site (RBS)
SEQ ID NO: 4
gcccgagtgagcacgc
CARE element AAV2
SEQ ID NO: 5
gtcctgtattagaggtcacgtgagtgctttgcgacattttgcgacaccatgtggtcacgctgggtacttaagcccga
gtgagcacgcagggtctccattttgaagcgggaggtttgaacgcgcagccgccatgccggggttttacgagattgtg
attaaggtacccagcgaccttgacgagcatctgcccggcatttctgacagctttgtgaactgggtggccgagaagga
atgggagttgccgccagattctgacatggatctgastctgattgagcaggcacccctgaccgtggccgagaagctgc
agcgcgactttctgacggaatggcgccgtgtgagtaaggccc
(UniProtKB - Q5TJ00)
L4 22K
Protein name: Human adenovirus D serotype 9 (HAdV-9)
SEQ ID NO: 6
MPRKKQEFLV EEMEEEWDSQ AEEDEWEEET EEEELEEVEE EQATEQPVAA
PSAPAAPAVT DTTSAAPAKP PRRWDRVKGD GKHERQGYRS WPAHKAAIIA
CLQDCGGNIA FARRYLLFHR GVNIFRNVLH YYRHLHS
Ad 5 L4 22K
SEQ ID NO: 7
atggcacccaaaaagaagctgcagctgccgccgccacccacggacgaggaggaatactgggacagtcaggcagagga
ggttttggacgaggaggaggaggacatgatggaagactgggagagcctagacgaggaagcttccgaggtcgaagagg
tgtcagacgaaacaccgtcaccctaggtcgcattcccctcgccggcgccccagaaatcggcaaccggttccagcatg
gctacaacctccgctcctcaggcgccgccggcactgcccgttcgccgacccaaccgtagatgggacaccactggaac
cagggccggtaagtccaagcagccgccgccgttagcccaagagcaacaacagcgccaaggctaccgctcatggcgcg
ggcacaagaacgccatagttgcttgcttgcaagactgtgggggcaacatctccttcgcccgccgctttcttctctac
catcacggcgtggccttcccccgtaacatcctgcattactaccgtcatctctacagcccatactgcaccggcggcag
cggcagcaacagcagcggccacacagaagcaaaggcgaccggatag
Ad 5 L4 22K
SEQ ID NO: 8
MAPKKKLQLPPPPTDEEEYWDSQAEEVLDEEEEDMMEDWESLDEEASEVEEVSDETPSPSVAFPSPAPQKSATGSSM
ATTSAPQAPPALPVRRPNRRWDTTGTRAGKSKQPPPLAQEQQQRQGYRSWRGHKNAIVACLQDCGGNISFARRFLLY
HHGVAFFPNILHYYRHLYSPYCTGGSGSNSSGHTEAKATG*
E2A polypeptide nucleotide sequence (Adenovirus type 5)
SEQ ID NO: 9
ATGGCCAGTCGGGAAGAGGagcagcgcgaaaccacccccgagcgcggacgcggtgcggcgcgacgtcccccaaccat
ggaggacgtgtcgtccccatccccgtcgccgccgcctccccgggcgcccccaaaaaagcggatgaggcggcgtatcg
agtccgaggacgaggsagactcatcacaagacgcgctggtgccgcgcacacccagcccgcggccatcgacctcggcg
gcggatttggccattgcgcccaagaagaaaaagaagcgcccttctcccaagcccgagcgcccgccatcaccagaggt
aatcgtggacagcgaggaagaaagagaagatgtggcgctacaaatgataggtttcagcaacccaccggtgctaatca
agcatggcaaaggaggtaagcgcacagtgcggcggctgaatgaagacgacccagtggcgcgtggtatgcggacgcaa
gaggaagaggaagagcccagcgaagcggaaagtgaaattacggtgatgaacccgctgagtgtgccgatcgtgtctgc
gtgggagaagggcatggaggctgcgcgcgcgctgatggacaagtaccacgtggataacgatctaaaggcgaacttca
aactactgcctgaccaagtggaagctctagcggccgtatgcaagacctggctgaacgaggagcaccgcaggttgcag
ctgaccttcaccagcsscaagacctttgtgacgatgatggggcgattcctgcaggcgtacatgcagtcgtttgcaga
ggtgacctacaagcatcacgagcccacgggctgcgcgttgtggctgcaccgctgcgctgagatcgaaggcgagctta
agtgtctacacggaagcattatgataaataaggagcacatgattgaaatggatgtgacgagcgaaaacgggcagcgc
gcgctgaaggagcagtatagcaaggccaagatagtgaagaaccggtggggccgaaatgtggtgcagatctccaacac
cgacgcaaggtgctgcgtgcacgacgcggcctgtccggccaatcagttttccggcaagtcttgcggcatgttcttct
ctcaaggcgcaaaggctcaggtggcttttaagcagatcaaggcttttatgcaggcgctctatcctaacgcccagacc
gggcacggtcaccttttgatgccactacggtgcgagtgcaactcaaagcctgggcacgcgccctttttgggaaggca
gctaccaaagttgactccgttcgccctgagcaacgcggaggacctggacgcggatctgatctccgacaagagcgtgc
tggccagcgtgcsccacccggcgcrgatagtgttccagtgctgcaaccctgtgtatcgcaactcgcgcgcgcagggc
ggaggccccaactgcgacttcaagatataggcgcccgacctgctaaacgcgttggtgatggtgcgcagcctgtggag
tgaaaacttcaccgagctgccgcggatggttatgcctgagtttaagtggagcactaaacaccagtatcgcaacgtgt
ccctgccagtggcgcatagcgatgcgcggcaGAACCCCTTTGATTTTTAA
ESA polypeptide amino acid sequence(Adenovirus type 5)
SEQ ID NO: 10
MASREEEQRETTPERGRGAARRFPTMEDVSSPSPSPPPFRAPPKKRMRRRIESEDEEDSSQDALVPRTPSFRPSTSA
ADLAIAPKKKKKRPSPKPERPPSPEVIVDSEEEREDVALQMVGFSNPPVLIKHGKGGKRTVRRLNEDDPVARGMRTQ
EEEEEPSEAESEITVMNPLSVPTVSAWEKGMEAARALMDKYHVDNDLKANFKLLPDQVEALAAVCKTWLNEEHRGLQ
LTFTSNKTFVTMMGRFLQAYLQSFAEVTYKHHEPTGCALWLHRCAEIEGELKCLHGSIMINKEHVIEMDVTSENGQR
ALKEQSSKAKIVKNRWGRNVVQISNTDARCCVHDAACPANQFSGKSCGMFFSEGAKAQVAFKQIKAFMQALYPNAQT
GHGHLLMPLRCECNSKPGHAPFLGRQLPKLTPFALSNAEDLDADLISDKSVLASVHHPALIVFQCCNPVYRNSPAQG
GGPNCDFKISAPDLLNALVMVRSLWSENFTELPPMVVPEFKWSTKHQYRNVSLPVAHSDARQNPFDF
TetR binding site
SEQ ID NO: 11
tccctatcag tgatagaga
Modified MLP
SEQ ID NO: 12
cgccctcttc ggcatcaagg aaggtgattg gtttgtaggt gtaggccacg tgaccgggtg
ttcctgaagg ggggctataa aaggtcccta tcagtgatag agactca
Modified MLP
SEQ ID NO: 13
cgccctcttc ggcatcaagg aaggtgattg gtttgtaggt gtaggccacg tgactcccta
tcagtgatag agaactataa aaggtcccta tcagtgatag agactca
Nucleotide sequence of the wild-type Ad5 MLP
SEQ ID NO: 14
cgccctcttcgggcatcaaggaaggtgattggtttataggtgtaggccacgtgaccgggtgttcctgaaggggggcta
taaaagggggtgggggcgcgttcgtcctca

### Sequence Listing Free Text

<210> 1

<213> Rep nucleotide sequence (adeno-associated virus 2)

<210> 2

<213> Cap nucleotide sequence (adeno-associated virus 2)

<210> 3

<213> Cap amino acid sequence (adeno-associated virus 2)

<210> 4

<223> Rep binding site (RBS)

<210> 5

<213> CARE element (adeno-associated virus 2)

<210> 6

<223> L4 22K (Human adenovirus D serotype 9 (HAdV-9))

<210> 7

<223> Ad 5 L4 22K

<210> 8

<223> Ad 5 L4 22K

<210> 9

<223> E2A polypeptide nucleotide sequence (Adenovirus type 5)

<210> 10

<223> E2A polypeptide amino acid sequence (Adenovirus type 5)

<210> 11

<223> TetR binding site

<210> 12

<223> Modified MLP

<210> 13

<223> Modified MLP

<210> 14

<223> Nucleotide sequence of the wild-type Ad5 MLP

1. A method of amplifying a DNA molecule in a host cell, wherein the DNA molecule is operably-linked to a CARE element, the method comprising the step of culturing a host cell which comprises:
(a) a first nucleic acid molecule comprising the DNA molecule operably-linked to a CARE element;
(b) a second nucleic acid molecule comprising a heterologous promoter operably-associated with a nucleotide sequence encoding a L4 22K polypeptide or a variant thereof;
(c) a third nucleic acid molecule comprising a nucleotide sequence encoding an AAV Rep polypeptide or a variant thereof;
and optionally additionally,
(d) one or more further nucleic acid molecules comprising one or more promoters operably-associated with one or more adenovirus Early gene products,
under conditions such that the second and third, and optionally additionally one or more of the further nucleic acid molecules, are expressed, thus promoting the amplification of the DNA molecule.

2. A method as claimed in claim 1, wherein the first, second, third and further (when present) nucleic acid molecules are independently present in the host cell:
(i) in an adenoviral vector;
(ii) stably integrated into the host cell genome; or
(iii) in an episomal vector or plasmid.

3. A method as claimed in claim 1, wherein the DNA molecule encodes a therapeutic polypeptide or a viral polypeptide.

4. A method as claimed in claim 3, wherein the DNA molecule encodes a rep gene sequence and/or a cap gene sequence and/or a viral Transfer Vector comprising flanking AAV inverted Terminal Repeats (ITRs), or a fragment thereof.

5. A process for producing virus particles, the process comprising the steps:
(a) introducing, into a host cell,
an adenoviral vector comprising:
(i) a nucleic acid molecule comprising a heterologous promoter operably-associated with a nucleotide sequence which encodes the L4 22K polypeptide or a variant thereof;
(ii) a Transfer Plasm id comprising 5′- and 3′-viral ITRs flanking a transgene;
(iii) sufficient helper genes for packaging a viral Transfer Plasmid,
the host cell comprising:
a CARE element, operably-linked to
(i) an AAV cap gene; and
(ii) a nucleic acid molecule comprising a nucleotide sequence encoding a viral Rep polypeptide;
(b) culturing the host cell under conditions such that virus particles are assembled within the host cell; and
(c) harvesting packaged virus particles from the host cell or from the culture medium.

6. A process for producing virus particles, the process comprising the steps:
(a) introducing, into a host cell,
an adenoviral vector comprising:
(i) a nucleic acid molecule comprising a heterologous promoter operably-associated with a nucleotide sequence which encodes the L4 22K polypeptide or a variant thereof;
(ii) a nucleic acid molecule comprising a nucleotide sequence encoding an viral Rep polypeptide,
(iii) sufficient helper genes for packaging a viral Transfer Plasmid,
the host cell comprising:
(i) a CARE element, operably-linked to an AAV cap gene; and
(ii) a Transfer Plasmid comprising 5′- and 3′-viral ITRs flanking a transgene, wherein the Transfer Plasmid may or may not be operably-linked to the CARE element,
stably integrated into the host cell genome;
(b) culturing the host cell under conditions such that virus particles are assembled within the host cell; and
(c) harvesting packaged virus particles from the host cell or from the culture medium.

7. A process as claimed in claim 5, wherein the AAV cap gene is integrated into the host cell genome under the control of a promoter that is activated by a polypeptide that is encoded within the adenoviral vector.

8. A process as claimed in claim 5, wherein the adenoviral vector comprises a repressible Major Late Promoter (MLP).

9. A process as claimed in claim 5, wherein the nucleotide sequence encoding the viral Rep polypeptide is inserted into the E1 region of an E1/E3-deleted adenoviral vector.

10. A process as claimed in claim 5, wherein the nucleic acid molecule comprising the nucleotide sequence encoding a viral Rep polypeptide does not comprise a functional p5 or a functional p19 promoter, and the nucleic acid molecule is not operably-associated with any other functional promoter, such that only baseline or minimal transcription of the Rep polypeptide-encoding sequence is obtained.

11. (canceled)

12. (canceled)

13. (canceled)

14. (canceled)

15. (canceled)

16. (canceled)

17. (canceled)

18. (canceled)

19. (canceled)

20. A process for producing virus particles as claimed in claim 5, wherein in Step (a), the nucleotide sequence encoding a viral Rep polypeptide is not operably-associated with a functional promoter.

21. A process for producing virus particles as claimed in claim 6, wherein the nucleotide sequence is not operably-associated with a functional promoter.

22. A process as claimed in claim 8, wherein the MLP comprises one or more repressor elements which are capable of regulating or controlling transcription of the adenoviral late genes, and wherein one or more of the repressor elements are inserted downstream of the MLP TATA box.
